(19) United States
(12) Patent Application Publication
(10) Pub. No.: US 2022/0175795 A1
(43) Pub. Date: Jun. 9, 2022

(54) USE OF FULVESTRANT FOR THE THERAPEUTIC CARE OF CENTRAL CORE DISEASE

(71) Applicants: UNIVERSITÉ GRENOBLE ALPES, Saint Martin d'Hères (FR); INSTITUT NATIONAL DE LA SANTE ET DE LA RECHERCHE MEDICALE, Paris (FR); CENTRE HOSPITALIER UNIVERSITAIRE GRENOBLE ALPES, La Tronche (FR); ASSOCIATION FRANCAISE CONTRE LES MYOPATHIES, Paris Cedex 13 (FR)

(72) Inventors: Isabelle MARTY, Veurey (FR); Laurent PELLETIER, Meylan (FR); Lauriane TRAVARD, Colombe (FR)

(21) Appl. No.: 17/604,719

(22) PCT Filed: Apr. 15, 2020
(86) PCT No.: PCT/IB2020/053549

§ 371 Date: Oct. 18, 2021

(30) Foreign Application Priority Data
Apr. 16, 2019 (EP) 19315023.2

(51) Int. Cl. A61K 31/565; A61P 21/00

(52) U.S. Cl. CPC A61K 31/565; A61P 21/00

(57) ABSTRACT

Disclosed is an estrogen receptor antagonist of formula:

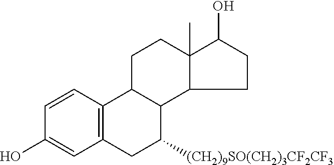

for use in the treatment of a disease or disorder related to a decrease in calcium release between the sarcoplasmic reticulum and the cytosol, in particular the disease or disorder is a myopathy related to one or more mutations of the RyR1 gene or a myopathy linked to a decrease in calcium release, and more particularly central core disease. Also disclosed is a pharmaceutical composition including at least the oestrogen receptor antagonist of and at least one pharmaceutically acceptable excipient.

### BACKGROUND OF THE INVENTION

### Field of the Invention

The present invention relates to the field of treatment of pathologies related to a reduction in calcium release, and in particular relates to the use of fulvestrant for the therapeutic care of central core disease.

Central core disease is a genetic disease characterized by a deficiency in muscle strength and a delay of muscle development. It is mainly associated with mutations in the type I ryanodine receptor (RyR1) gene. RyR1 is in reality a calcium channel allowing the release of calcium from the sarcoplasmic reticulum towards the cytosol. In the cytosol, calcium induces the slippage of myofibrils leading to muscle contraction.

The importance of RyR1 in the cascade of events leading from the stimulation of the muscle fiber by the motor neuron to its contraction perfectly explains that a mutation in the corresponding gene, altering its expression or function, induces a defect in muscle strength.

Indeed, the contraction of the skeletal muscle includes several steps. Stimulation from the motor neuron first causes depolarization of the plasma membrane of the muscle fiber. This depolarization propagates to triads, structures consisting of an invagination of the plasma membrane, the T-tubule, associated with two terminal cisterns of sarcoplasmic reticulum. There, it activates the dihydropyridine receptor (DHPR) located in the membrane of the T-tubule, which in turn activates the type 1 ryanodine receptor (RyR1), to which it is mechanically coupled. This second calcium channel located in the membrane of the sarcoplasmic reticulum is responsible for the discharge (or release) of calcium from the reticulum towards the cytosol. This calcium release out of the intracellular stocks finally allows the myosin filaments to slide over the actin filaments, and thus allows muscle contraction. The calcium is then pumped back to the reticulum.

### Description of the Related Art

At the present time, there is no specific treatment for the care of patients with central core disease. The only care provided to patients are kinesitherapy and corticoids to improve the general condition, but there is no curative treatment.

The current research mainly relates to the development of gene therapies aimed at correcting the mutation or its immediate consequences. The problem lies mainly in the fact that there are many mutations of RyR1 linked to central core disease, therefore, each gene therapy strategy cannot therefore be directed to a particular patient or a small number of patients.

Research published in 2013 (Dorchies et al, 2013) demonstrated the therapeutic properties of tamoxifen in mice representing a model of Duchenne dystrophy. In addition to improving the strength of these mice once treated, the authors have noted numerous histological and functional modifications without however being able to explain the efficiency of this agent. The same reasearch group also addressed the therapeutic effect of tamoxifen on another muscular disease, myotubular myopathy.

Duchenne dystrophy and myotubular myopathy are not closely related, but in both cases, alterations in calcium release were observed (Luca et al., 2001, Plant et al., 2003, Dowling et al. 2009 and 2010). The mechanistic lead is not experimentally addressed or even mentioned in the publication of Dorchies et al.

### SUMMARY OF THE INVENTION

The present inventors have then developed a new therapeutic strategy for their pathology of interest, central core disease, starting from the principle that tamoxifen is a specific regulator of estrogen receptors. For this purpose, it modulates the effect that the estrogens have on many tissues. However, the effect of tamoxifen is complex because it sometimes exerts an antagonistic effect on tissues (breast) and sometimes agonist (uterus, bone). Its effect is not characterized at the muscle level.

To overcome the uncertainty of the agonist/antagonist effect of tamoxifen on muscle tissue, the inventors have brought their attention to fulvestrant, a pure estrogen receptor antagonist.

Fulvestrant is a known molecule, prescribed at the current time in the context of supporting hormone-dependent breast cancers. It has, in this context, the same activity as tamoxifen.

In the present invention, the therapeutic strategy consists in seeking to improve the calcium release from the sarcoplasmic reticulum towards the cytosol which is a determining step for muscle contraction. The use of fulvestrant in the therapeutic care of central core disease constitutes the solution identified by the inventors to compensate the lack of specific treatment of this myopathy.

The inventors dispose of a murine model of central core disease that they have developed in the laboratory and characterized. This model is based on a research for new broad spectrum therapeutic strategies, as opposed to gene therapies based on the correction of the mutations that are so numerous that they are almost specific to each patient suffering from central core disease.

The interest in the therapeutic strategy of the present invention makes it possible to address a wide population of patients, the agent targeting a common mechanism, regardless of the mutation responsible for the decrease in RyR1.

The inventors have noted that the literature refers to tamoxifen, an estrogen receptor modulator which improves the symptoms of two muscle pathologies, namely Duchenne dystrophy and myotubular myopathy, without the authors having been able to elucidate the mechanisms responsible for these improvements. As these pathologies present some characteristics shared with central core disease, the inventors have evaluated this treatment.

Estrogen receptors are intracellular proteins of the steroid receptor family which therefore possess a transcription factor function. There are two types of estrogen receptors, ERα and ERβ, encoded by two different genes: ESR1 and ESR2, respectively. The alternative splicing of the mRNA is responsible for several isoforms for each type of receptor. Their expression profile depends on the tissue. The variable relative expression of the isoforms, that of the partner factors participating in the activation or inhibition of transcription, as well as the existence of independent effects of the receptors, render the estrogene signalling particularly complex (Kulkoyluoglu et al, 2016). The action of tamoxifen is complex: it acts as an estrogen receptor agonist or antagonist in a tissue-dependent manner (Berry et al., 1990), depending in particular on the proportions in which the two receptors are expressed. It has indeed an agonist action through ERα, and an antagonist action through ERβ (Salvatori et al., 2003).

The action of tamoxifen being complex, the inventors have decided to bring their attention to an agent having a simpler mode of action. They therefore focused on a pure estrogen receptor ERα and ERβ antagonist, fulvestrant. They then have administered fulvestrant to mice in which they have induced a central core disease and observed that despite the loss of weight associated with the development of the disease, the mice did not lose strength over the entire duration of the treatment (75 days). They also have carried out in vitro studies on primary murine satellite cells of skeletal muscles, as well as on immortalized human cells derived from a biopsy of a patient suffering from central core disease.

### DETAILED DESCRIPTION OF THE INVENTION

The present invention relates to the estrogen receptor antagonist of formula:

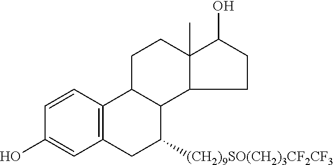

for use in the treatment of a disease or disorder related to a decrease in calcium release between the sarcoplasmic reticulum and the cytosol.

This antagonist is in particular known as fulvestrant.

In one embodiment, the disease or disorder may be a myopathy related to one or more mutations in the RyR1 gene or a myopathy related to a decrease in calcium release.

In a particular embodiment, the disease or disorder may be central core disease.

Central core disease is a neuromuscular disorder characterized by round lesions at the center of the muscle fiber, visible on the muscle biopsy, and by the clinical expression of congenital myopathy.

In one aspect, the antagonist is intended for administration to a subject in a therapeutically effective amount.

The expression “therapeutically effective amount” means the amount or quantity of compound necessary and sufficient to slow down or stop the progression, aggravation or deterioration of one or more symptoms of the disease or disorder, in particular a disease or disorder related to a decrease in calcium release, in particular a myopathy related to one or more mutations in the RyR1 gene, more particularly central core disease; alleviating the symptoms of the disease or disorder, in particular a myopathy related to one or more mutations of the RyR1 gene, more particularly central core disease.

The “therapeutically effective amount” depends on the subject, the stage of the disease to be treated and the administration method, and can be determined by routine operations by a person skilled in the art. This amount may vary with age and gender of the subject.

Advantageously, a therapeutically effective amount can vary between 0.01 and 100 mg/kg body mass, preferably between 0.1 and 20 mg/kg, and more preferably between 1 and 10 mg/kg, for example in one or more weekly administrations, for one or more months.

In particular, the therapeutically effective human amount can be between 50 mg/month and 500 mg/month for a woman of 60 kg, most preferably the therapeutically effective human amount is 250 mg/month for a woman of 60 kg, or of the range of 4.17 mg/kg/month.

By way of example, for mice, the therapeutically effective amount has been adapted according to the recommendation of the FDA of the United States (Food and Drug Administration), namely by multiplying the therapeutically effective human amount by a factor 12.3 to obtain the effective murine amount. Thus, the preferred murine dose is 51 mg/kg/month, or for a 20 g mouse, a dose of 1 mg/month. In the context of the present invention and for the tests performed on mice, an amount of about 0.25 mg/week was administered by intramuscular injection.

In addition, the therapeutically effective amount specific for any patient will depend on a variety of factors including the treated disorder and the severity of the disorder; the activity of the specific compound used; the specific composition used, age, body mass, general health status, gender and diet of the patient; the duration of administration, the administration route, and the excretion rate of the specific compound used; the duration of the treatment; the drugs used in combination or simultaneously with the specific compound used; and the similar factors well known in the medical technique. For example, it is well in the skills of a person skilled in the art to begin dosing the compound at levels lower than those required to achieve the desired therapeutic effect and to gradually increase the dosage until the desired effect is achieved.

According to another aspect, the antagonist of the present invention is intended for use as a medicine.

The invention also relates to a pharmaceutical composition comprising:
at least the estrogen receptor antagonist of the present invention;
at least one pharmaceutically acceptable excipient.

The expression “pharmaceutically acceptable excipient” refers to non-toxic material that is compatible with a biological system such as a cell, cell culture, tissue or organism. This pharmaceutically acceptable excipient does not produce an undesirable, allergic or other reaction when administered to an animal, in particular a human. The characteristics of the excipient will depend on the administration mode used.

This includes any solvent, diluent, dispersion medium, binder, linker, lubricant, disintegrant, coating, antibacterial and antifungal agent, isotonic agent and absorption retarding agent, and the like. A pharmaceutically acceptable excipient refers to a non-toxic solid, semi-solid or liquid filler, a diluent, an encapsulating material or an accessory formulation of any type. For human administration, the preparations should meet the requirements of sterility, pyrogenicity, general safety and purity as required by the good manufacturing practices of the active substances for human and veterinary use.

In one aspect, the pharmaceutical composition is intended for administration to a subject in a therapeutically effective amount.

In the pharmaceutical compositions of the present invention, the active ingredient, alone or in combination with another active ingredient, can be administered in a unit administration form, in the form of a mixture with conventional pharmaceutical carriers, to animals and humans. Suitable unit administration forms include oral dosage forms such as tablets, capsules, powders, granules and suspensions or solutions for oral route, sublingual and buccal administration forms, aerosols, implants, subcutaneous, transdermal, topical, intraperitoneal, intramuscular, intravenous, subcutaneous, transdermal intrathecal and intranasal administration forms, and rectal administration forms.

According to another aspect, the pharmaceutical composition is intended for use as a medicine.

To this end, the pharmaceutical composition or the medicament contains vehicles which are pharmaceutically acceptable for a formulation suitable for oral administration.

Exemplary forms suitable for oral administration include, but are not limited to, tablets, orodispersible tablets, effervescent tablets, powders, granules, pills (including sweetened pills), dragees, capsules (including soft gelatin capsules), syrups, liquids, gels or other solutions, suspensions, slurries, liposomal forms and the like.

In one embodiment, the pharmaceutical composition or medicament contains vehicles which are pharmaceutically acceptable for a formulation capable of being injected.

Examples of suitable injection forms include, but are not limited to, solutions, such as, for example, sterile aqueous solutions, dispersions, emulsions, suspensions, solid forms suitable for use in preparing solutions or suspensions by adding a liquid prior to use, for example, a powder, liposomal forms, or the like.

The delivery mode may be by injection or by gradual infusion. The injection may be intravenous, intra-peritoneal, intramuscular, subcutaneous or transdermal.

The preparations for parenteral administration can include sterile aqueous or non-aqueous solutions, suspension or emulsions. Examples of non-aqueous solvents are benzyl alcohol, ethanol, propylene glycol, polyethylene glycol, vegetable oils or injectable organic esters such as ethyl oleate. Aqueous vehicles include water, alcohol/water solutions, emulsions or suspensions.

The invention also relates to a method for treating a disease or disorder related to a decrease in calcium release between the sarcoplasmic reticulum and the cytosol, in particular a myopathy linked to one or more mutations of the RyR1 gene or a myopathy related to a decrease in calcium release, more particularly central core disease.

The method of treatment of the present invention comprises administering to a subject in need thereof a therapeutically effective amount of the estrogen receptor agonist of formula:

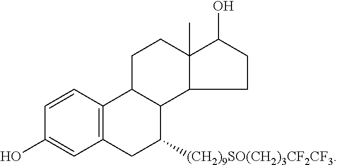

In another embodiment, the method of treatment of the present invention comprises administering to a subject in need thereof a therapeutically effective amount of the pharmaceutical composition of the invention.

The invention thus relates to a method for treating a patient suffering from a disease or disorder related to a decrease in the release of calcium between the sarcoplasmic reticulum and the cytosol, in particular a myopathy linked to one or more mutations in the RyR1 gene or a myopathy related to a decrease in calcium release, more particularly the central core disease, comprising administering a therapeutically effective amount of the estrogen receptor agonist of the invention or the pharmaceutical composition of the invention.

The administration of the estrogen receptor agonist or the pharmaceutical composition of the invention can be carried out by any above-mentioned administration route.

In order to better illustrate the subject matter of the present invention, we will now describe below, by way of illustration and in a non-limiting manner, the following examples in connection with the attached drawings.

### BRIEF DESCRIPTION OF THE DRAWINGS

In these drawings:

FIG. 1 is a diagram of the construction of the inducible RyR1 KO mouse model.

FIG. 2 is a graphic representation of the fluorescence measurements in calcium imaging on murine cells.

FIG. 3A is a graphic representation of fluorescence measurements in calcium imaging on murine cells, in the presence or absence of treatment with fulvestrant 100 nM.

FIG. 3B is a bar graph representing the magnitude of the fluorescence peak in calcium imaging, in the presence or absence of treatment with fulvestrant 100 nM.

FIG. 4A is a graphic representation of the fluorescence measurements in calcium imaging on human control cells CTRL, in the presence or absence of treatment with fulvestrant 100 nM.

FIG. 4B is a graphic representation of the fluorescence measurements in calcium imaging on human control cells CTRL, in the presence or absence of treatment with fulvestrant 250 nM.

FIG. 4C is a graphic representation of the fluorescence measurements in calcium imaging on immortalized human MELA cells derived from a biopsy of a patient suffering from central core disease, in the presence or absence of treatment with fulvestrant 100 nM.

FIG. 4D is a graphic representation of the fluorescence measurements in calcium imaging on immortalized human MELA cells derived from a biopsy of a patient suffering from central core disease, in the presence or absence of treatment with fulvestrant 250 nM.

FIG. 4E is a bar graph representing the magnitude of the fluorescence peak in calcium imaging on human control cells CTRL, in the presence or absence of treatment with fulvestrant 100 nM or 250 nM.

FIG. 4F is a bar graph representing the magnitude of the fluorescence peak in calcium imaging on human MELA cells, in the presence or absence of treatment with fulvestrant 100 nM or 250 nM.

FIG. 5 is a graph representing the strength of the post-induction mice of central core disease as measured in gripping time over time, in the presence or absence of treatment with fulvestrant 100 nM.

### DESCRIPTION OF THE PREFERRED EMBODIMENTS

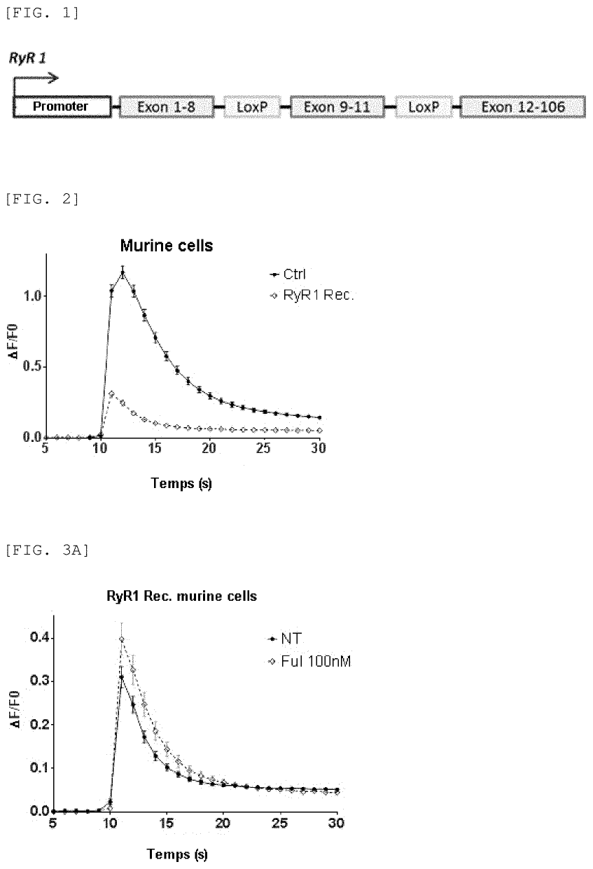

In FIG. 1, the schematization of the genetic construct of the inducible RyR1 KO mouse model developed by the inventors can be seen. Exons 9-11 of the RYR1 gene are flanked by LoxP sites, on both alleles of the gene. The mice also have a HSA-Cre/ERT2 transgene encoding Cre recombinase. Inactivation of the gene by KO is induced by intraperitoneal injections of tamoxifen. Thus, in the presence of tamoxifen, the Cre recombinase is translocated towards the nucleus and the Cre-Lox recombination system makes it possible to carry out a deletion of exons 9-11 of the RYR1 gene. This laboratory-developed inducible KO mouse model is non-lethal and constitutes a model for studying central core disease.

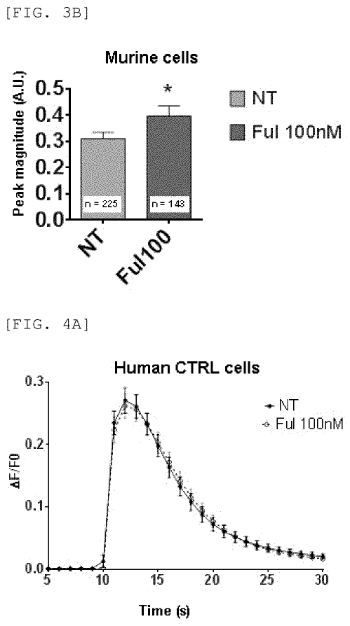

Referring to FIG. 2, in calcium imaging, the cells are loaded for 30 minutes with the Fluo-4 probe which, by fixing Ca²⁺ ions, emits fluorescence proportional to the cytoplasmic concentration of calcium. The calcium releases from the sarcoplasmic reticulum towards the cytosol are therefore directly followed on live cells by video-microscopy after stimulation by KCl at 140 mM (mimick a membrane depolarization). Thus, we can see that on murine cells in primary culture, the induction of the inactivation of the KO of the RYR1 gene (recombinant RyR1 cells) results in a significant reduction of the calcium release peak, which reduction was quantified at 73% in comparison to the release of calcium in the control murine cells (Ctrl).

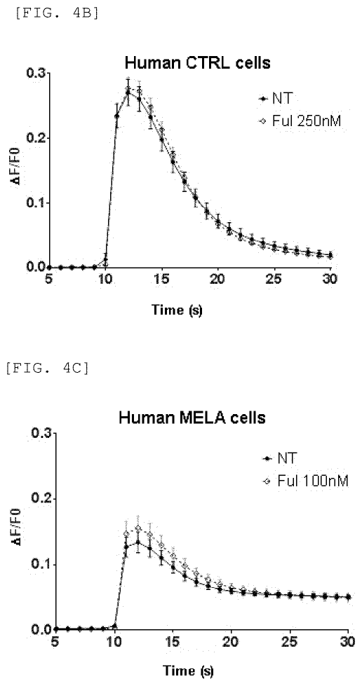

In a similar manner, complementary studies in calcium imaging were conducted on these murine cells in primary culture, after induction of recombination by AdV-Cre transduction (recombinant RyR1 cells). Thus, it is possible to observe in FIG. 3A that the fulvestrant at 100 nM improves calcium release with respect to untreated cells (NT). FIG. 3B shows the magnitude of the calcium release peak, which passes from 0.31+/−0.02 to 0.40+/−0.04, or an increase of 28% (Student Test, p<0.05). The number of myotubes analyzed in each condition is indicated in the bars of the graph.

The same experiments were carried out on control human muscle cells CTRL and on MELA cells, which are immortalised human cells derived from a biopsy of a patient having central core disease.

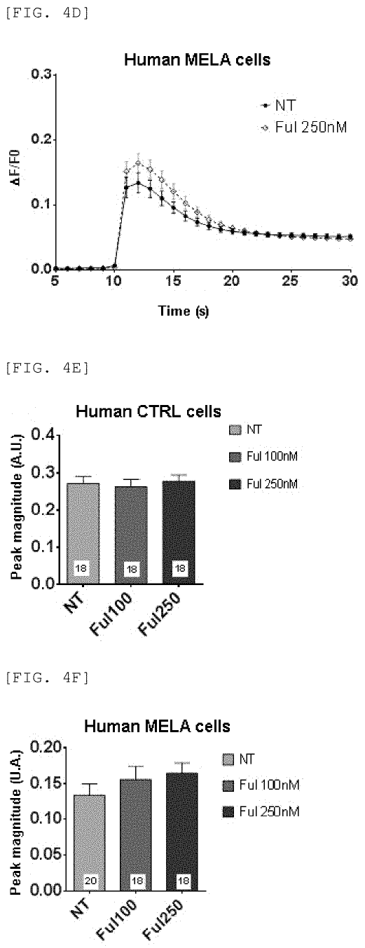

Referring to FIG. 4A, it can be seen that the fulvestrant treatment at 100 nM does not have an effect on the control cells. Similarly, the treatment with fulvestrant at 250 mM does not have any effect on the control cells (FIG. 4B).

On the other hand, it can be seen in FIG. 4C that the calcium release peak of human cells MELA is significantly reduced compared to control cells, whether the cells are treated by fulvestrant 100 nM or not. It can be noted that the treatment with fulvestrant 100 nM tends to increase calcium release, but in a non-statistically significant manner. The same observation can be made from FIG. 4D: the release of calcium has collapsed in the human cells MELA and the treatment with fulvestrant 250 nM also tends to increase the release of calcium relative to the untreated cells, but the statistical data is not shown to be significant.

Referring to FIG. 4E, the graph shows that the treatment with fulvestrant, whether at a concentration of 100 nM or 250 nM, has no effect on the control human muscle cells. The number of wells analyzed in each condition is indicated in the bars of the graph.

On the other hand, if reference is made to FIG. 4F, the graph makes it possible to visualize the tendency to improve the calcium release of human cells MELA in response to a fulvestrant treatment at 100 nM and even more by the fulvestrant at 250 nM. It is also noted that the calcium release peak has passed from about 0.28 in human muscle cells to about 0.15 in immortalized cells derived from a biopsy from a patient with central core disease.

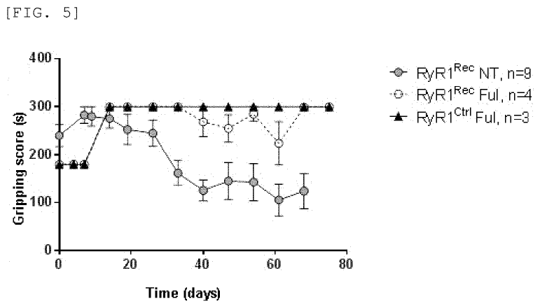

Finally, if reference is made to FIG. 5, it can be seen that the gripping time of mice in which the central core disease (RyR1^{Rec}) treated with fulvestrant 100 nM is improved with respect to untreated mice (NT). The strength of the mice treated with fulvestrant is significantly improved compared to untreated mice.

### Examples

The following examples illustrate the invention.

Materials and Methods

Animal Model

For this study, an inducible and non-lethal model of KO mice for RyR1 developed in the laboratory was used. In these mice, exons 9 to 11 of the RYR1 gene are flanked by LoxP sites on both alleles (RYR1 fl/fl). These mice also have a HSA-Cre/ERT2 transgene (RyR1^{Rec}) coding for Cre recombinase, whereas this construct is absent in control mice (RyR1^{Ctrl}).

KO was induced by intraperitoneal injections of tamoxifen (Sigma) eight weeks after birth, at 1 mg/day for five days. It is important to note that all animals have received these injections, whether they have the HSA-Cre allele or not.

Exclusively in animals having the HSA-Cre allele, tamoxifen (TAM) causes the translocation of the Cre recombinase to the nucleus, thus allowing the recombination of the LoxP sites and the deletion of exons 9-11 from the RYR1 gene (FIG. 1). In order to improve the understanding, the mice in which the recombination cannot be made will be referred to as the RyR1^{Ctrl} mice in the following document. The recombinant mice after intraperitoneal administration of TAM will be referred to as RyR1^{Rec} mice.

Animal Treatments

For the treatment of animals, fulvestrant was prepared from a commercial solution of Faslodex 250 mg (AstraZeneca). This solution was diluted in physiological serum and 0.27 mg of fulvestrant was injected to each mouse (weight of 20 g) in 50 μl volume. The injections were carried out intramuscularly in the quadriceps once per week.

The choice of the injected dose was made in accordance with the recommendation of the FDA indicating to use a dose 12.3 times greater than that of the recommended dose in humans.

Here, for a human dose of 250 mg/month in a woman of 60 kg, the dose applied to the weight in the mouse is 51 mg/kg/month, or 1 mg/month for a 20 g mouse, distributed in 4 injections, therefore about 0.25 mg/week.

Animal Grip Test

In order to evaluate the muscular strength of the animals, the mice were placed on a grid which was then turned upside down. The duration during which the mice remain gripped at the grid has been timed to a limit of 300 seconds. This measurement was carried out in a weekly manner over the entire population of mice of the study.

Cell Cultures

Primary Murine Satellite Cells

The satellite cells were isolated from the skeletal muscles of the lower limbs of the neonate mice RyR1^{Ctrl} according to the procedure described in Marty et al, 2000.

The cells were seeded in 96 well plates (75,000 cells/well) previously coated with laminin. The cells were first cultured in the proliferation medium composed of Ham's F-10 Nutrient Mix (Life Technologies), 20% fetal calf serum (Life Technologies), 2% Ultroser G (PALL) and 2% Penicillin-Streptomycin (Life Technologies).

The RyR1^{Ctrl} cells thus produced do not express the Cre recombinase. In order to induce floxed RyR1 gene recombination, the cells thus have been transduced 12 hours after seeding with adenoviruses allowing the expression of Cre recombinase, AdV-Cre (Utah University), to a multiplicity of infections (multiplicity of infection, or MOI) of 64.

24 hours after seeding, the cells were placed in the differentiation medium composed of Dulbecco's Modified Eagle Medium (Life Technologies), 2% of horse serum (Life Technologies) and 1% of Penicillin-Streptomycin (Life Technologies) for 3 days.

Human Cells

The CTRL cells are immortalized human cells from a biopsy of a healthy subject having no calcium release abnormality.

The MELA cells are immortalized human cells derived from a biopsy of a patient suffering from central core disease, including in particular a quantitative defect of RyR1 and a calcium release defect (Rendu et al., 2013). These cells were immortalised by the team of Vincent Mouly (Institute of Myology, Paris).

The cells were seeded in 96 well plates (50 000 cells/well) in the growth medium of composition identical to that of the primary murine cells.

24 hours after seeding, the cells were placed in the differentiation medium for 7 days. The medium has been renewed 4 days after placing in the differentiation medium.

Culture Treatment

Fulvestrant (Tocris) was solubilized in dimethyl sulfoxide at a concentration of 10 mg/ml. This stock solution was maintained at −20° C. and used for cellular tests after dilution in the cell differentiation medium at the desired concentration. The cells have been treated throughout the duration of the differentiation.

Calcium Imaging

The cells were loaded for 30 minutes with Fluo-4 Direct (Thermofisher). Calcium releases are followed by a video-microscope Leica DMI6000 FRAP (Leica Microsystems) for a duration of 40 seconds, with stimulation with KCl at 140 mM.

The films were analyzed by the Fiji (Schindelin et al., 2012) and Prim (GraphPad) softwares.

For murine cells, the fluorescence measurements were performed on regions of interest each corresponding to a myotube, with several tens of myotubes measured for each repetition of the experiment.

For human cells, the shape of the cells not allowing them to be distinguished individually in an accurate manner, the measurements were made on a single region of interest per well corresponding to the total surface covered by the myotubes. 4 to 6 wells were measured for each repetition of the experiment.

Statistical Analyses

The comparisons were made with the Student test using Prim software (GraphPad). The differences are considered to be significant when p<0.05.

### Example 1: Study of the Discharge (or Release) of Calcium In Vitro on Primary Murine Cells

The RyR^{Ctrl} cells transduced or not by Cre recombinase AdV-Cre have been treated with the various agents, from the differentiation time to the time of functional analysis. The intensity of the calcium release was evaluated in response to a stimulation by KCl 140 mM (mimicking a depolarization at ˜0 mV).

As expected, the expression of Cre-recombinase is accompanied by the reduction of the calcium release capabilities of myotubes. Indeed, as shown in FIG. 2, the induction of the inactivation by KO of the RyR1 gene is accompanied by a reduction of 73% of the amplitude of the calcium release peak (p<0.01).

Murine RyR^{CTRL} cells after induction of recombination by AdV-Cre transduction (RyR1-Rec cells) are placed in differentiation medium in the absence (NT) or in the presence of fulvestrant 100 nM (Ful 100 nM). After 3 days of differentiation, calcium releases were studied in calcium imaging (FIG. 3A). Fulvestrant at 100 nM improves calcium release, the peak passes from 0.31+/−0.02 to 0.40+/−0.04, or an increase of 28% (p<0.05) (FIG. 3B).

### Example 2: Study of the Discharge (or Release) of Calcium In Vitro on Human Cells

Fulvestrant was tested on human muscle cells having or not a calcium release defect (human cells CTRL or MELA), by incubation of 7 days in differentiation medium.

As shown in FIGS. 4A and 4B, the fulvestrant has no effect on the control cells CTRL. In MELA cells, the calcium releases of which are collapsed, fulvestrant tends to increase calcium release both at a concentration of 100 nM and at 250 nM (FIGS. 4C-4E). However, the quantification being made no more on each myotube but on each well of the culture plate, the number of samples is lower and the difference does not reach the statistical significance.

### Example 3: Measurement of the In Vivo Strength in Mice Treated with Fulvestrant

Female mice (RyR1^{CTRL} mouse or RyR1^{Rec} mouse) have been treated either by fulvestrant 100 nM (Ful) or by physiological serum (NT) in intramuscular injection once a week throughout the duration of the experiment.

The strength of the animals is followed by a grip test (gripping test) once a week, just before the injections. The results of the strength measurement over time are presented in FIG. 5.

The results obtained on this small group of fulvestrant-treated female mice show a very sharp improvement in mouse strength compared to untreated mice. Statistical analyses may be performed on a larger number of animals, also including male individuals.

### BIBLIOGRAPHIC REFERENCES

Berry M, Metzger D, Chambon P, Role of the two activating domains of the oestrogen receptor in the cell-type and promoter-context dependent agonistic activity of the antioestrogen 4-hydroxytamoxifen, EMBO J. 1990 September; 9(9): 2811-2818.
De Luca A, Pierno S, Liantonio A, Cetrone M, Camerino C, Simonetti S, Papadia F, Camerino D C, Alteration of excitation-contraction coupling mechanism in extensor digitorum longus muscle fibres of dystrophic mdx mouse and potential efficacy of taurine, Br J Pharmacol. 2001 March; 132(5):1047-54.
Dorchies O M, Reutenauer-Patte J, Dahmane E, Ismail H M, Petermann O, Patthey-Vuadens O, Comyn S A, Gayi E, Piacenza T, Handa R J, Décosterd L A, Ruegg U T, The anticancer drug tamoxifen counteracts the pathology in a mouse model of duchenne muscular dystrophy, Am J Pathol. 2013 February; 182(2):485-504.
Dowling J J, Low S E, Busta A S, Feldman E L, Zebrafish MTMR14 is required for excitation-contraction coupling, developmental motor function and the regulation of autophagy, Hum Mol Genet. 2010 Jul. 1; 19(13):2668-81.
Dowling J J, Vreede A P, Low S E, Gibbs E M, Kuwada J Y, Bonnemann C G, Feldman E L, Loss of myotubularin function results in T-tubule disorganization in zebrafish and human myotubular myopathy, PLoS Genet. 2009 February; 5(2):e1000372.
Kulkoyluoglu E, Madak-Erdogan Z, Nuclear and extranuclear-initiated estrogen receptor signaling crosstalk and endocrine resistance in breast cancer, Steroids. 2016 October; 114:41-47.
Plant D R, Lynch G S, Depolarization-induced contraction and SR function in mechanically skinned muscle fibers from dystrophic mdx mice, Am J Physiol Cell Physiol. 2003 September; 285(3):C522-8.
Rendu J, Brocard J, Denarier E, Monnier N, Piétri-Rouxel F, Beley C, Roux-Buisson N, Gilbert-Dussardier B, Perez M J, Romero N, Garcia L, Lunardi J, Faure J, Fourest-Lieuvin A, Marty I, Exon skipping as a therapeutic strategy applied to an RYR1 mutation with pseudo-exon inclusion causing a severe core myopathy, Hum Gene Ther. 2013 July; 24(7):702-13.

Salvatori L, Pallante P, Ravenna L, Chinzari P, Frati L, Russo M A, Petrangeli E, Oestrogens and selective oestrogen receptor (ER) modulators regulate EGF receptor gene expression through human ER alpha and beta subtypes via an Sp1 site, Oncogene, 2003 Jul. 31; 22(31):4875-81.
Schindelin, J.; Arganda-Carreras, I. & Frise, E. et al. (2012), Fiji: an open-source platform for biological-image analysis, Nature methods 9(7): 676-682.

1. A method of treatment of a disease or disorder related to a decrease in calcium release between the sarcoplasmic reticulum and the cytosol, comprising administering a therapeutically effective dose of estrogen receptor antagonist of formula: 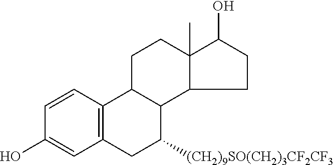
to a patient in need thereof.

2. The method of claim 1, wherein the disease or disorder is a myopathy related to one or more mutations in the RyR1 gene or a myopathy related to a decrease in calcium release.

3. The method of claim 1, wherein the disease or disorder is central core disease.

4-5. (canceled)

6. A pharmaceutical composition comprising:
an estrogen receptor antagonist of formula: 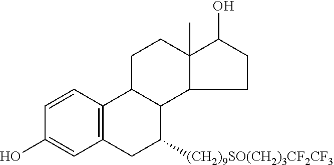
at least one pharmaceutically acceptable excipient.

7. The pharmaceutical composition according to claim 6, wherein the composition is suitable to be administered to a subject in a therapeutically effective amount.

8. The pharmaceutical composition according to claim 6, wherein the composition is suitable for use as a medicine.

9. The method of claim 2, wherein the disease or disorder is central core disease.

10. The pharmaceutical composition according to claim 7, wherein the composition is suitable for use as a medicine.
